(19) United States
(12) Patent Application Publication
(10) Pub. No.: US 2025/0346553 A1
(43) Pub. Date: Nov. 13, 2025

(54) Process for the Preparation of R-Oxybutynin Hydrochloride

(71) Applicant: HARMAN FINOCHEM LIMITED, Mumbai, Maharashtra (IN)

(72) Inventors: Nitin Sharadchandra Pradhan, Thane (West), Maharashtra (IN); Vijay Trimbak Kadam, Aurangabad, Maharashtra (IN); Dhananjay Uddhavrao Edaki, Aurangabad, Maharashtra (IN); Ravindra Bhausaheb Pagire, Ahmednagar, Maharashtra (IN); Tukaram Sarjerao Choudhare; Ganesh Machhindra Chavan, Ahmednagar, Maharashtra (IN); Harpreet Singh Minhas, Mumbai, Maharashtra (IN); Gurpreet Singh Minhas

(21) Appl. No.: 18/862,265

(22) PCT Filed: Apr. 27, 2023
(86) PCT No.: PCT/IN2023/050414

§ 371 Date: Nov. 1, 2024

(30) Foreign Application Priority Data
May 5, 2022 (IN) 202221026193

(51) Int. Cl. C07C 213/06

(52) U.S. Cl. CPC C07C 213/06; C07C 2601/14; C07C 2601/16

(57) ABSTRACT

The present invention discloses a method for resolving cyclohexylphenyl glycolic acid and the preparation of optically active phenylcyclohexyl glycolate esters. More particularly, the invention provides a process for preparation of optically active R-oxybutynin hydrochloride with high enantiomeric purity of greater than 99%.

### FIELD OF THE INVENTION

The present invention relates to a method for resolving cyclohexylphenyl glycolic acid and the preparation of optically active cyclohexylphenyl glycolate esters. More particularly, the invention provides a process for preparation of optically active R-oxybutynin hydrochloride with high enantiomeric purity of greater than 99%.

### BACKGROUND OF THE INVENTION

The U.S. Pat. No. 3,176,019 discloses oxybutynin hydrochloride and its synthesis wherein, the methyl phenyl cyclohexyl glycolate is reacted with 4-diethylamino-2-butynylacetate in presence of a base to yield oxybutynin followed by treatment with HCl solution to form hydrochloride salt.

Cyclohexylphenyl glycolic acid (also referred to herein as “CHPGA”) is used as a starting material for manufacturing oxybutynin (4-diethylamino-2-butynyl phenylcyclohexylglycolate) and its metabolite, such as desethyloxybutynin (4-ethylamino-2-butynyl phenylcyclohexylglycolate). The(S)-enantiomers of oxybutynin and its metabolite, desethyloxybutynin have been shown to provide a superior therapy in treating urinary incontinence, as disclosed in U.S. Pat. Nos. 5,532,278 and 5,677,346. Further, the (R) enantiomer of oxybutynin has also been suggested to be a useful drug candidate, as reported in J. Pharmacol. Exp. Ther. 256, 562-567 (1991)).

Racemic CHPGA is generally prepared by one of the two methods: (1) selective hydrogenation of phenyl mandelic acid or of phenyl mandelate esters, as shown in Scheme 1;

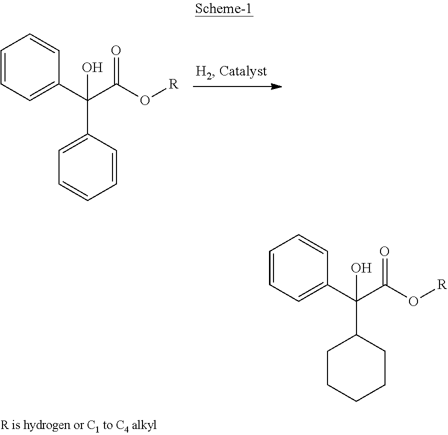

or (2) Grignard addition reaction of cyclohexyl magnesium halide to phenylglyoxylate as shown in Scheme 2.

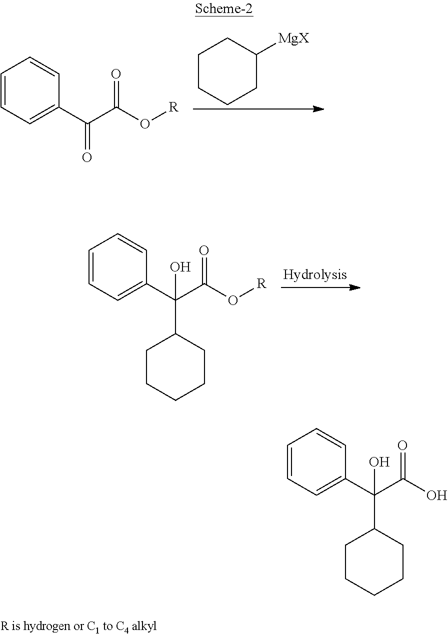

Asymmetric synthesis of individual enantiomers of CHPGA are prepared by Grignard addition to a chiral auxiliary ester of glyoxylic acid to give a diastereomeric mixture of esters, as per Scheme 2. In addition, a multi-step asymmetric synthesis of (R)-CHPGA from (D)-arabinose using alkyl or phenyl Grignard reagents has also been reported.

Kachur et al. in J. Pharmacol. Exper. Ther: 247, 867-872 (1988) reported a process which is shown in Scheme 3 below, wherein, R* in the chiral ester is the residue of a chiral alcohol, that can be directly converted into chiral enantiomers of Oxybutynin by trans-esterification (R′=acetate), or hydrolysed to yield chiral CHPGA (R′═H).

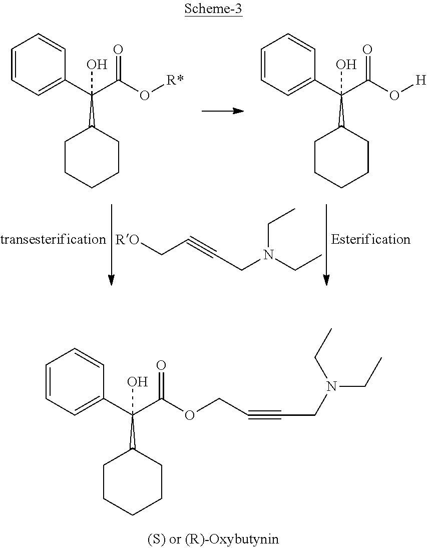

U.S. Pat. No. 6,140,529 discloses a process for preparing a single enantiomer of cyclohexylphenyl glycolic acid by a) providing a solution comprising racemic cyclohexylphenyl glycolic acid and a single enantiomer of tyrosine methyl ester; b) driving a salt of primarily one diastereomer out of Solution; c) separating the diastereomeric salt from the solution; and d) liberating the single enantiomer of cyclohexylphenyl glycolic acid from the diastereomeric salt.

U.S. Pat. No. 6,140,529 further discloses a process for preparation of single enantiomer of Oxybutynin. The synthetic method directly couples an enantiomer of CHPGA with a propargyl alcohol derivative to produce a single enantiomer of a propargyl ester of cylclohexylphenylglycolate. The method comprises:
(a) activating the carboxy group of a single enantiomer of cyclohexylphenyl glycolic acid to form an activated carboxy compound; and
(b) adding to the activated carboxy compound a side chain propargyl alcohol derivative of structure (III), to produce the single enantiomer of structure (II).

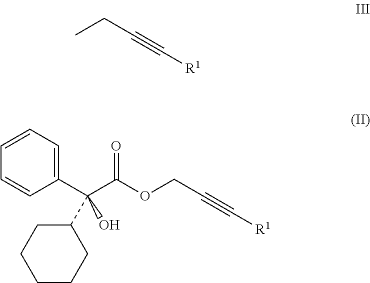

Though U.S. Pat. No. 6,140,529 described the process for preparing a single enantiomer of cyclohexylphenyl S-Oxybutynin hydrochloride, however, there is no details provided about separation and isolation of R-CHPGA which is key intermediate for the synthesis of R-Oxybutynin hydrochloride. Moreover, the reported yields are around 85%.

The aforementioned asymmetric synthetic methods reported in the prior arts suffer from poor chemical yields and the stereoselectivity is not always high. Also, the chiral auxiliary reagents that give good yields and higher stereoselectivity are often quite expensive and thus makes the process industrially not viable

Therefore, there remains a need in the art for the process for separation and isolation of R-Oxybutynin hydrochloride having high chiral purity and high yield which can overcome the drawbacks of the prior arts processes. In pursuit of the above, the present inventors have surprisingly found an efficient process for the preparation of R-Oxybutynin hydrochloride which offer great advantages over the prior art processes in terms of high yield, high chiral purity and generates less effluents and further simple and scalable procedure suitable for large scale industrial production of R-Oxybutynin hydrochloride.

### SUMMARY OF THE INVENTION

The present invention provides a novel process for preparing optically active R-oxybutynin hydrochloride with high enantiomeric purity of greater than 99%, which process comprises the following steps;
a) Reacting D-Valine with thionyl chloride in the presence of an alcohol to obtain the corresponding D-Valine alkyl ester;
b) Reacting the D-Valine alkyl ester with racemic cyclohexyl phenyl glycolic acid (CHPGA) in the presence of water and acetonitrile to obtain R-CHPGA D-valine alkylester;
c) Hydrolysing the R-CHPGA D-Valine alkyl ester to obtain pure R-CHPGA;
d) Reacting the R-CHPGA with 4-(Diethylamino) but-2-yn-1-ol in the presence of ethylchloroformate & an organic base to obtain R-oxybutynin hydrochloride; and
e) optionally purifying the R-oxybutynin hydrochloride obtained in step (d) with isopropyl alcohol and diisopropyl ether to obtain pure R-Oxybutynin hydrochloride.

### DETAILED DESCRIPTION OF THE PRESENT INVENTION

The invention will now be described in detail in connection with certain preferred and optional embodiments, so that various aspects thereof may be more fully understood and appreciated.

Unless specified otherwise, all technical and scientific terms used herein have the same meaning as commonly understood by one of ordinary skill in the art, to which this invention belongs.

The present invention provides a novel process for preparing optically active R-oxybutynin hydrochloride with high enantiomeric purity of greater than 99%, which process comprises the following steps;
a) reacting D-Valine with thionyl chloride in the presence of an alcohol to obtain D-Valine alkylester;

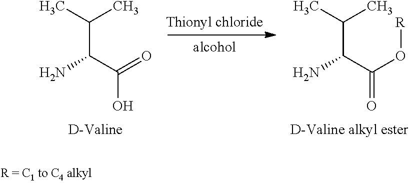

b) reacting the D-Valine alkyl ester with racemic cyclohexyl phenyl glycolic acid (CHPGA) in the presence of water and acetonitrile to obtain R-CHPGA D-valine alkyl ester;

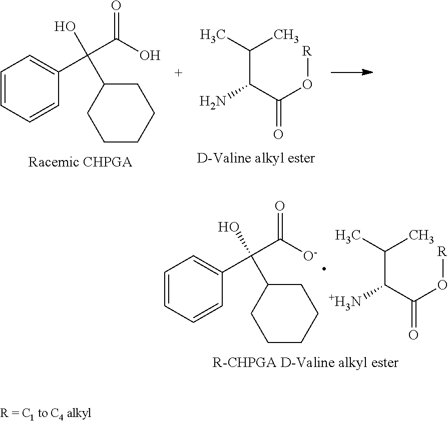

c) hydrolysing R-CHPGA D-Valine alkyl ester to obtain pure R-CHPGA;

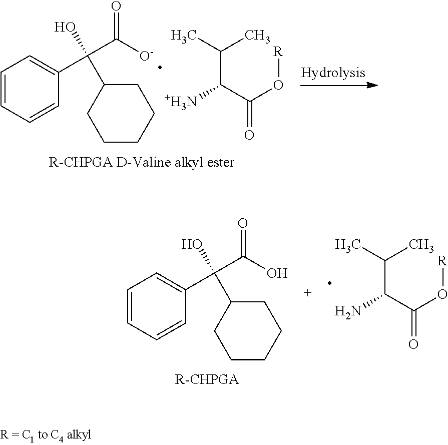

d) reacting the R-CHPGA with 4-(Diethylamino) but-2-yn-1-ol in the presence of ethylchloroformate & an organic base to obtain R-oxybutynin hydrochloride; and

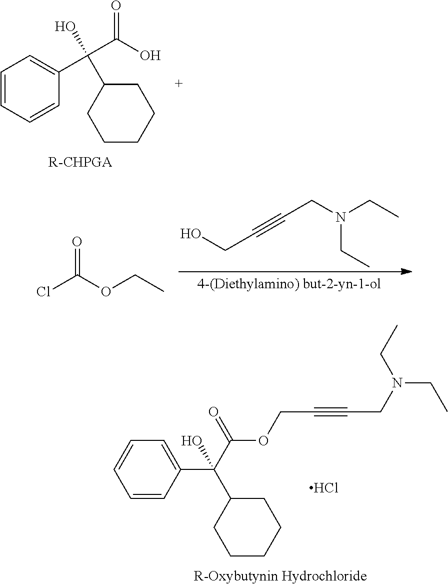

e) optionally purifying the R-oxybutynin hydrochloride obtained in step (d) with isopropyl alcohol and diisopropyl ether to obtain pure R-Oxybutynin hydrochloride.

In an embodiment, the alkyl esters of D-valine can be prepared by using appropriate alcohols of C₁ to C₄ carbon atoms, for example, D-Valine methyl ester can be prepared by using methanol; D-Valine ethyl ester can be prepared by using ethanol; D-Valine propyl ester can be prepared by using propyl alcohol and D-Valine butyl ester can be prepared by using butyl alcohol.

In an embodiment, the organic base is selected from the group consisting of triethylamine, diethylamine, diisopropylamine, pyridine, pyrolidine and piperidine.

The entire process for the synthesis of R-oxybutynin hydrochloride can be conducted at a temperature range of room temperature to reflux temperature of the solvent used.

Accordingly, in one of the embodiments, D-Valine was reacted with methanol in presence of thionyl chloride at 40-50° C. for upto 20 to 30 hrs. After completion of the reaction methanol was distilled out completely and acetone was added to the reaction mass and the reaction mass was cooled to 0-5° C. Filtered the solid & dried at 50-60° C. to obtain D-Valine methyl ester.

In an alternate embodiment, D-valine ethyl ester and D-valine isopropyl esters were prepared by reacting D-valine with ethanol and isopropanol respectively in presence of thionyl chloride. The esters thus obtained were used in subsequent chemical reactions to obtain R-Oxybutynin hydrochloride.

In the next stage, R-Cyclohexyl phenyl glycolic acid D-Valine methyl ester was synthesised by treating racemic cyclohexyl phenyl glycolic acid with D-Valine methyl ester in presence of acetonitrile and methanol at a pH range of 7 to 9 at 50-60° C. and after completion of the reaction by TLC, the mass was cooled to 30-40° C. and filtered the solid, which was purified from a mixture of acetonitrile and water to obtain R-Cyclohexyl phenyl glycolic acid D-Valine methyl ester.

In the next stage, R-Cyclohexyl phenyl glycolic acid (R-CHPGA) was prepared by hydrolysing the R-phenyl Cyclohexyl glycolic acid D-Valine methyl ester in water by adjusting the pH of the reaction mass to 2.0 to 3.0 with a mineral acid and isolated the solid obtained, washed the solid with water and dried at 40-50° C., to obtain crude R-Cyclohexyl phenyl glycolic acid (R-CHPGA).

In alternate embodiments, the R-Cyclohexyl phenyl glycolic acid (R-CHPGA), can also be obtained by reacting racemic cyclohexyl phenyl glycolic acid with other D-Valine alkyl esters under similar conditions as explained above, for example, D-Valine ethyl ester; D-Valine propyl ester or D-Valine butyl ester to obtain R-Cyclohexyl phenyl glycolic acid D-Valine ethyl ester; R-Cyclohexyl phenyl glycolic acid D-Valine propyl ester; or R-Cyclohexyl phenyl glycolic acid D-Valine butyl ester respectively, and subsequent hydrolysis of the same.

In the final stage, the crude R-oxybutynin hydrochloride was prepared by reacting (R)-Cyclohexyl-phenylglycolic acid with 4-(Diethylamino) but-2-yn-1-ol in presence of ethyl chloroformate and an organic base at 70-90° C. for 10 to 20 hrs. The product was separated from the organic layer and the organic layer was taken in water, the pH was adjusted to 1.0-2.0 with dil. HCl at 45-55° C., and separated the layers. The aqueous layer was basified to pH 8.0 to 9.0 and extracted the product, R-Oxybutynin into an organic solvent, distilled the solvent to obtain the crude R-Oxybutynin, which was taken in isopropyl alcohol and pH was adjusted to 3.0-4.0 by IPA. HCl, to isolate crude R-Oxybutynin hydrochloride.

In yet another embodiment, the crude R-oxybutynin hydrochloride was purified from isopropyl alcohol and further crystallised from Diisopropyl ether to obtain pure R-Oxybutynin hydrochloride.

The process of the present invention results in R-Oxybutynin hydrochloride having a chiral purity of greater than 99.5%.

The following examples are presented to further explain the invention with experimental conditions, which are purely illustrative and are not intended to limit the scope of the invention.

### EXAMPLES

### Example-1: Preparation of D-Valine Methyl Ester from D-Valine

Thionyl chloride (165 ml) was slowly added to the reaction mass containing D-Valine (100 g) and methanol (400 ml) at 0-5° C. The reaction mass was then heated to 40-50° C. & maintained for 25 hrs. After reaction completed Methanol is distilled out completely and 100 ml acetone was added to the reaction mass. The acetone was distilled out completely. Further acetone (200 ml) was added. The reaction mass was cooled to 0-5° C. Filtered to isolate solid & dried at 50-60° C. to obtain the title compound (130 g).

### Example-2: Synthesis of R-Cyclohexyl Phenyl Glycolic Acid D-Valine Methyl Ester

In a clean and dry RBF acetonitrile (1000 ml) racemic cyclohexyl phenyl glycolic acid (CHPGA-100 g) was added. The reaction mass was heated to 50-60° C. D-Valine Methyl Ester solution (50 g in 20 ml methanol having pH 7.0-9.0) was added. The reaction mass was stirred for 1 hr at 50-60° C. The reaction mass was cooled to 30-40° C. The reaction mass was filtered and the solid was isolated. The obtained solid was charged in acetonitrile (1300 ml) and water (10 ml). The reaction mixture was heated to 60-70° C. and maintained for 1 hr and cooled to 30-40° C. Filtered to isolate the solid Yield-75 gm

Chiral purity by HPLC—99.50%

### Example-3: Preparation of R-Cyclohexyl Phenyl Glycolic Acid (R-CHPGA)

Purified water (800 ml) and R-phenyl Cyclohexyl glycolic acid D-Valine methyl ester (100 g) was charged in a RBF. Adjusted pH to 2.0-3.0 by hydrochloric acid. The reaction mass was further stirred for 30 min. The obtained solid was filtered & washed with 100 ml purified water. Solid was filtered and dried at 40-50° C. to obtain the title compound (70.0 g).

Chiral purity by HPLC—99.5%

### Example-4: Preparation of Crude R-Oxybutynin Hydrochloride

(R)-Cyclohexyl-phenylglycolic acid is charged to Heptane (600 ml) in a RBF and stirred at 25-35° C. Cool the reaction mass 15-25° C. and slowly charged triethyl amine (57.5 g) and ethyl chloroformate (37.5 g). The reaction mass was stirred for 3 hours. 4-(Diethylamino) but-2-yn-1-ol (42.5 g) was added to the reaction mass. The reaction mass was heated to 70-90° C. and maintained for 12 hrs. The reaction mass was cooled to 25-35° C. and added water (375 ml). Separated the layers and organic layer was washed with sodium chloride solution. The organic layer was taken in water (75 ml) and heated to 45-55° C. pH was adjusted to 1.0-2.0 with dil. HCl, stirred for 30 min, and separated the layers. The aqueous layer was basified to pH 8.0 to 9.0 with 20% sodium hydroxide solution. The product was extracted in methylene dichloride. The methylene chloride layer is distilled out atmospherically and degas under reduced pressure. Isopropyl alcohol (50 ml) was added, stirred for 15-20 min and distilled out under reduced pressure. Further charged isopropyl alcohol (300 ml) at 25-35° C. followed by pH adjustment to 3.0-4.0 by IPA·HCl. The reaction mass stirred for complete precipitation. Filtered the solid & washed with isopropyl alcohol (50 ml) and dried at 45-55° C. for 10 hr to obtain crude R-Oxybutynin hydrochloride (50 g-70 g).

Chiral Purity by HPLC—: 99.6%

### Example-5: Purification of R-Oxybutynin Hydrochloride

Crude R-oxybutynin hydrochloride (100 g) was taken in isopropyl alcohol (300 ml) and heated to 50-60° C. to obtain the clear solution. Activated charcoal is added and stirred the reaction mass for 30 min. Filtered the mass through hyflobed and collected the filtrate. The filtrate was cooled to 20-30° C. and stirred to achieve complete crystallization. Filtered the solid & washed with isopropyl alcohol (50 ml) and dried at 45-55° C. Dried material was taken in Diisopropyl ether (1000 ml) and stirred at 60-70° C. for 3 hrs. The reaction mass was cooled to 25-30° C. and maintained for 2 hrs. Filtered and washed the wet cake with Diisopropyl ether (100 ml) and dried at 45-55° C. to obtain pure R-Oxybutynin hydrochloride (95 g).

Chiral Purity by HPLC—99.8%

### Example-6: Preparation of D-Valine Ethyl Ester from D-Valine

Thionyl chloride (165 ml) was added slowly in the reaction mass of D-Valine (100 g) and ethanol (400 ml) at 0-5° C. The reaction mass was then heated to 40-50° C. and maintained for 25 hrs. After completion of the reaction ethanol is distilled out completely. Acetone was added to the reaction mass & cool the reaction mass to 0-5° C. stirred for 3 hr. Filtered and dried the product at 50-60° C. to obtain the titled compound (126 g)

### Example-9: Preparation of D-Valine Propyl Ester from D-Valine

Thionyl chloride (165 ml) was added slowly in the reaction mass of D-Valine (100 g) and n-propanol (400 ml) at 0-5° C. The reaction mass was then heated to 40-50° C. and maintained for 25 hrs. After completion of the reaction n-propanol is distilled out completely. Acetone was added to the reaction mass & cool the reaction mass to 0-5° C. stirred for 3 hr. Filtered and dried the product at 50-60° C. to obtain the titled compound (130 g)

1. A novel process for preparing an optically active R-oxybutynin hydrochloride with an enantiomeric purity of greater than 99%, wherein the process comprises the following steps;
a) reacting valine with thionyl chloride in the presence of an alcohol to obtain valine alkyl ester; 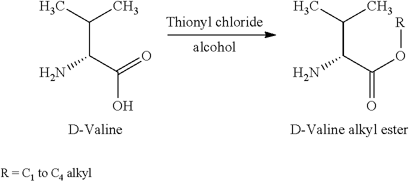
b) reacting the valine alkyl ester with racemic cyclohexyl phenyl glycolic acid (CHPGA) in the presence of water and acetonitrile to obtain R-CHPGA D-valine alkyl ester; 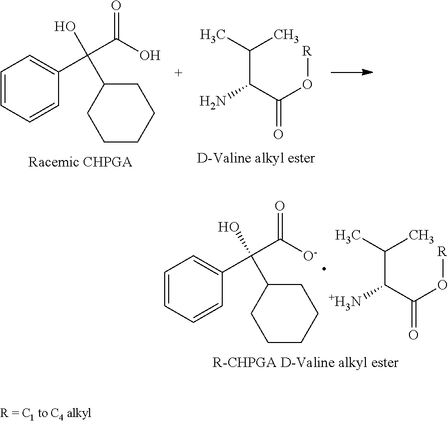
c) hydrolysing R-CHPGA valine alkyl ester to obtain pure R-CHPGA; 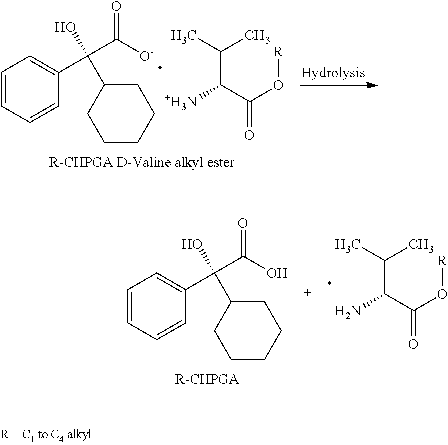
d) reacting the R-CHPGA with 4-(diethylamino) but-2-yn-1-ol in the presence of ethylchloroformate and an organic base to obtain R-oxybutynin hydrochloride; and 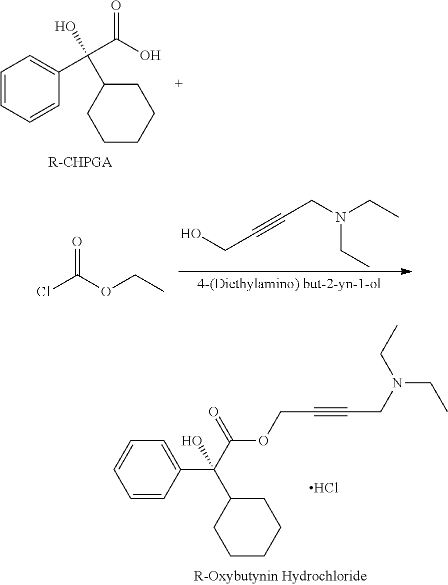
e) optionally purifying the R-oxybutynin hydrochloride obtained in step (d) with isopropyl alcohol and diisopropyl ether to obtain pure R-oxybutynin hydrochloride.

2. The process as claimed in claim 1, wherein, the alcohol in step a) is a C₁ to C₄ alcohol selected from the group consisting of methanol, ethanol, isopropanol, and butanol.

3. The process as claimed in claim 1, wherein; the valine alkyl ester is selected from the group consisting of valine methyl ester, valine ethyl ester, valine propyl ester, and valine butyl ester.

4. The process as claimed in claim 1, wherein; the R-CHPGA D-valine alkyl ester is selected from the group consisting of R-CHPGA D-valine methyl ester, R-CHPGA D-valine ethyl ester, R-CHPGA D-valine propyl ester, and R-CHPGA D-valine butyl ester.

5. The process as claimed in claim 1, wherein; the organic base used in step d) is selected from the group consisting of triethylamine, diethylamine, diisopropylamine, pyridine, pyrrolidine, and piperidine.

6. The process as claimed in claim 1, wherein, the process is conducted at a temperature of room temperature to reflux temperature of the solvent used.

7. The process as claimed in claim 1, comprising the step of purifying the R-oxybutynin hydrochloride obtained in step (d) with isopropyl alcohol and diisopropyl ether to obtain pure R-oxybutynin hydrochloride.

8. The process as claimed in claim 1, wherein the alcohol in step a) is methanol.

9. The process as claimed in claim 1, wherein the D-valine alkyl ester is D-valine methyl ester.

10. The process as claimed in claim 1, wherein the R-CHPGA D-valine alkyl ester is R-CHPGA D-valine methyl ester.

11. The process as claimed in claim 1, wherein the organic base used in step d) is triethylamine.

12. The process as claimed in claim 1, wherein the alcohol in step a) is methanol, the D-valine alkyl ester is D-valine methyl ester, and the R-CHPGA D-valine alkyl ester is R-CHPGA D-valine methyl ester.

13. The process as claimed in claim 12, wherein the organic base used in step d) is triethylamine.

14. The process as claimed in claim 1, wherein the alcohol in step a) is ethanol, the D-valine alkyl ester is D-valine ethyl ester, and the R-CHPGA D-valine alkyl ester is R-CHPGA D-valine ethyl ester.

15. The process as claimed in claim 1, wherein the alcohol in step a) is propanol, the D-valine alkyl ester is D-valine propyl ester, and the R-CHPGA D-valine alkyl ester is R-CHPGA D-valine propyl ester.

16. The process as claimed in claim 1, wherein the optically active R-oxybutynin hydrochloride has an enantiomeric purity of 99.5% or more.

17. The process as claimed in claim 1, wherein the optically active R-oxybutynin hydrochloride has an enantiomeric purity of greater than 99.5%.
